(19) United States
(12) Patent Application Publication
(10) Pub. No.: US 2016/0030528 A1
(43) Pub. Date: Feb. 4, 2016

(54) ANTIMICROBIAL MURAMIDASE

(71) Applicant: VANDERBILT UNIVERSITY, Nashville, TN (US)

(72) Inventors: Jason METCALF, Antioch, TN (US); Seth BORDENSTEIN, Nashville, TN (US)

(73) Assignee: VANDERBILT UNIVERSITY, Nashville, TN (US)

(21) Appl. No.: 14/731,167

(22) Filed: Jun. 4, 2015

Related U.S. Application Data
(60) Provisional application No. 62/019,652, filed on Jul. 1, 2014.
(60) Provisional application No. 62/108,921, filed on Jan. 28, 2015.

(51) Int. Cl. A61K 38/47

(52) U.S. Cl. CPC A61K 38/47; C12Y 302/01017

(57) ABSTRACT

Aciduliprofundum boonei glycosyl hydrolase 25 (GH25) muramidase is shown here to exhibit antibacterial activity against several distinct bacterial families. Formulations and methods of use for this GH25 are provided.

This application claims benefit of priority to U.S. Provisional Application Ser. Nos. 62/019,652, and 62/108,921, filed Jul. 1, 2014, and Jan. 28, 2015, respectively. The entire contents of each application are hereby incorporated by reference.

This invention was made with government support under Grant Number RO1 GM085163 awarded by the National Institutes of Health. The government has certain rights in the invention.

### BACKGROUND OF THE INVENTION

1. Field of the Invention

This disclosure relates to the fields of microbiology and infectious disease. More particularly, the disclosure relates to the use of a GH25 muramidase to kill bacteria, e.g., to treat bacterial infections.

2. Related Art

Genome-enabled studies indicate that horizontal gene transfers (HGTs) experience a frequency gradient that decreases from: within domain>between two domains>between all domains of life. Within the domain Bacteria, HGT is rampant among prokaryotes and phages and is an important mechanism for acquisition of new genes and functions (Popa & Dagan, 2011). HGT is well known for shuttling antibiotics and antibiotic resistance between bacteria (Clardy et al., 2009). Between two domains, instances of horizontal transfer of diverse genes are well documented, including transfer between bacteria and archaea in the species Thermatoga maritima (Nelson et al., 1999), widespread transfer between bacterial endosymbionts and their hosts (Husnik et al., 2013), movement of genes between rotifers and fungi, plants, and bacteria (Gladyshev et al., 2008), transfer between eukaryotes and their viruses (Bratke & McLysaght, 2008), and HGT between bacteria and nematode parasites of plants (Danchin et al., 2010). Although some of these transfers have been functionally characterized, the biological activity, selective advantages, and ecological contexts of many interdomain HGT events remain poorly characterized (Dunning-Hotopp, 2008; Keeling & Palmer, 2008).

In the case of transfer of one gene to all cellular domains of life, the parallel movement of the same gene family is quite rare. Among the few putative cases, there is a pore-forming toxin domain that appears to have been anciently transferred between diverse lineages (Moran et al., 2012). However, the distribution of the transfer across the tree of life is unclear because archaea sequences were not included in this study's phylogenetic analyses due to low support values. A second class spans genes present since the last common ancestor whose evolution typically includes vertical descent and ancient HGTs. These genes can encode nucleotide metabolism, intramembrane proteolysis, or membrane transport, but the transfers have not been functionally validated in recipient taxa. In addition, the transfer events can be challenging to characterize due their deep antiquity in evolutionary time and the confounding issues of ancient paralogy (Lundin et al., 2010; Koonin et al., 2003; McClure, 2001; McDonald et al., 2012). It would thus appear that recurrent gene transfer to multiple domains of life is extremely uncommon and subject to very rare events throughout the history of life.

One significant question is why do single interdomain transfers occur more frequently than recurrent transfers to multiple domains? There are at least two explanations. First, recurrent transfer of the same gene family may be limited by incompatible mechanics of gene transfer (e.g., transduction, transfection, plasmid exchange) between domains compared to within domains. However, the individual success of gene transfer between any two domains of life suggests that this barrier may be minimal. Second, once a gene is transferred between organisms, the selective barriers against HGT of the same gene family are multifaceted given that each new donor-recipient combination may or may not benefit from the trait conferred in the transfer. Thus, there may be very few of what are termed “spreader” genes that repeatedly increase fitness of the recipient across the whole diversity of life. Given the extent of antibiotic-related HGT in bacteria, the transfer of antibacterial genes to other domains of life could bestow similar selective advantages to nonbacterial taxa inhabiting a bacterial world.

### SUMMARY OF THE INVENTION

Thus, in accordance with the present disclosure, there is provided a pharmaceutical composition comprising an Aciduliprofundum boonei glycosyl hydrolase 25 muramidase (GH25) domain disposed in a pharmaceutically acceptable diluent, carrier or excipient. The muramidase domain may have a sequence according to SEQ ID NO: 1, or have a sequence that is about 70%, about 80%, about 90%, or about 95% homologous to the sequence according to SEQ ID NO: 1, or have a sequence that hybridizes under high stringency conditions to a sequence encoding SEQ ID NO: 2. The muramidase may include a lysozyme sequence according to SEQ ID NO: 3. The muramidase domain may be fused to a non-Aciduliprofundum boonei sequence, such as a purification tag, including a 6×-His tag. The pharmaceutical composition may further comprise a distinct anti-bacterial agent, such as a second anti-bacterial peptide or a chemical antibiotic. The composition may comprise an imidazole.

In another embodiment, there is provided an expression cassette encoding an Aciduliprofundum boonei glycosyl hydrolase 25 muramidase (GH25) domain, operably linked to a promoter. The muramidase domain may have a sequence according to SEQ ID NO: 1, or have a sequence that is about 70%, about 80%, about 90%, or about 95% homologous to the sequence according to SEQ ID NO: 1, or have a sequence that hybridizes under high stringency conditions to a sequence according to SEQ ID NO: 2. The muramidase may include a lysozyme sequence according to SEQ ID NO: 3. The muramidase domain may be fused to a non-Aciduliprofundum boonei sequence, such as a purification tag, including a 6×-His tag. The promoter may be a prokaryotic promoter or a eukaryotic promoter.

In still another embodiment, there is provided a replicable vector encoding an Aciduliprofundum boonei glycosyl hydrolase 25 muramidase (GH25) domain. The muramidase domain may have a sequence according to SEQ ID NO: 1, or have a sequence that is about 70%, about 80%, about 90%, or about 95% homologous to the sequence according to SEQ ID NO: 1, or have a sequence that hybridizes under high stringency conditions to a sequence according to SEQ ID NO: 2. The muramidase may include a lysozyme sequence according to SEQ ID NO: 3.

In a further embodiment, there is provided a cell comprising a nucleic acid segment encoding an Aciduliprofundum boonei glycosyl hydrolase 25 muramidase (GH25) domain, wherein said cell is not an Aciduliprofundum boonei cell. The muramidase domain may have a sequence according to SEQ ID NO: 1, or have a sequence that is about 70%, about 80%, about 90%, or about 95% homologous to the sequence according to SEQ ID NO: 1, or have a sequence that hybridizes under high stringency conditions to a sequence according to SEQ ID NO: 2. The muramidase may include a lysozyme sequence according to SEQ ID NO: 3.

In still a further embodiment, there is provided a method of inhibiting a bacterium comprising contacting said bacterium with an Aciduliprofundum boonei glycosyl hydrolase 25 muramidase (GH25) domain. The muramidase domain may have a sequence according to SEQ ID NO: 1, or have a sequence that is about 70%, about 80%, about 90%, or about 95% homologous to the sequence according to SEQ ID NO: 1, or have a sequence that hybridizes under high stringency conditions to a sequence according to SEQ ID NO: 2. The muramidase domain may include a lysozyme sequence according to SEQ ID NO: 3. The method may further comprise contacting said bacterium with a distinct anti-bacterial agent, such as a second anti-bacterial peptide or a chemical antibiotic. The bacterium may be from the phylum Firmicutes, including the family Bacillaceae or Paenibacillaceae, such as Bacillus subtilis, Bacillus megaterium, Paenibacillus polymyxa, or the phylum Proteobacteria, such as Eschericia coli. The bacterium may be located in a biological material ex vivo. The bacterium may be located in a living subject, such as a human or non-human animal subject. The bacteria may be located on a surface of a machine, device or article of manufacture. The machine may be a food processing/handling machine, or a heating/cooling/ventilation machine. The device may be a medical device, a food preparation or service device, or an animal cage or stall. The method may further include any use of the muramidase in disinfecting or sterilizing any environment (e.g., surgical suites) or product (e.g., foodstuffs). The method may further comprise providing an imidazole in combination with a GH25 domain.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed.

The patent or application file contains at least one drawing executed in color. Copies of this patent or patent application publication with color drawing(s) will be provided by the Office upon request and payment of the necessary fee.

FIG. 1. HGT candidates and surrounding genes. Each arrow represents an open reading frame transcribed from either the plus strand (arrow pointing right) or the minus strand (arrow pointing left). The color of the arrow indicates the taxa the gene is found in based on its closest homologs. Black=Eubacteria, purple=virus, red=Archaea, green=Plantae, Orange=Fungi, Blue=Insecta, white=no known homologs, dashed line=present in multiple domains. The length of the arrows and intergenic regions are drawn to scale except where indicated with broken lines. The four paralogs of the lysozyme in S. moellendorffii occur on two genomic scaffolds with light green bands connecting homologous genes. Abbreviations: Lys: lysozyme, gpW=phage baseplate assembly protein W, SH3: Src homology domain 3, App-1=ADP-ribose-1″monophosphatase, PRT=phosphoribosyltransferase, LD=leucoanthocyanidin dioxygenase; IMP=integral membrane protein. A protein diagram for each lysozyme is drawn to scale with the light gray regions highlighting a conserved protein domain. *A. pisum diagram is based on Acyr_{—}1.0 assembly and transcription data (Nikoh et al., 2010); the annotation in Acyr_{—}2.0 is different.

FIGS. 2A-D. Phylogeny of GH25 muramidase. (FIG. 2A) Phylogeny based on alignment of 113aa without indels consisting of top E-value hits to blastp using WORiA phage lysozyme as a query. Taxon of origin for each amino acid sequence is indicated by color. Posterior probability (Bayesian phylogeny) and bootstrap values (maximum likelihood phylogeny) are indicated at all nodes with values above 50. Branch lengths represent number of substitutions per site as indicated by scale bar. Tree is arbitrarily rooted. Iterative phylogenies based on top E-value blastp hits to A. boonei lysozyme (FIG. 2B), A. pisum lysozyme (FIG. 2C), and S. moellendorffii lysozyme (FIG. 2D) are also shown.

FIGS. 3A-C. Conservation and selection of A. boonei GH25 muramidase domain. (FIG. 3A) Consensus alignment of 86 GH25 muramidases with insertions and deletions removed. Conservation is indicated by amino acid symbol size and bar graphs below the consensus sequence (SEQ ID NO: 5). Active site residues and highly conserved amino acids modeled below are indicated with red and orange asterisks, respectively. Space-filling model of the (FIG. 3B) active site face and (FIG. 3C) 180° turn of predicted structure of A. boonei GH25 muramidase domain. Active site residues are indicated in red, the eight additional residues most highly conserved across all 86 proteins are orange, and residues that may be under positive selection are blue.

FIGS. 4A-B. Antibacterial action of A. boonei GH25 muramidase domain against Firmicutes. (FIG. 4A) Bacteria of the specified strain/species incubated overnight on tryptic soy agar after a 20-minute liquid preincubation with the proteins indicated. Genera: B=Bacillus, P=Paenibacillus. Proteins: CEWL=chicken egg white lysozyme, P. poly=P. polymyxa lysozyme, PhiBP=bacteriophage PhiBP lysozyme, A. boo=GH25 domain of A. boonei lysozyme, CFP=cyan fluorescent protein. Images are representative of at least three independent experiments. (FIG. 4B) Dose-dependence of A. boonei GH25 muramidase antibacterial action. B. subtilis colony survival after incubation with A. boonei GH25 muramidase at the indicated concentrations for 20 min at 37° C. N=10 for each concentration. P<0.001 for linear model fit. Error bars are +/−SEM.

FIGS. 5A-B. Presence of HGT lysozyme genes in field samples. (FIG. 5A) PCR amplifications of portions of the GH25 muramidase domain in the indicated taxa. All amplifications were Sanger sequenced to confirm integration. Primers used are listed in Table 3. Abbreviations: Sb: S. braunii, Sm: S. moellendorffii, Su: S. uncinata, Ssa: S. sanguinolenta, Sst: S. stauntoniana, Sl: S. lepidophylla, E: East Pacific Rise, L: Lao Spreading Center, M: Mid-Atlantic Ridge, Pu: Pleotrichophorus utensis, Aa: Artemisaphis artemisicola, Ue: Uroleucon erigeronensis, Av: Aphis varians, Ap: Acyrthosiphon pisum, Al: Aphis lupini, Cs: Cedoaphis sp., As: Aphthargelia symphoricarpi, Bs: Braggia sp., -: water only control. (FIG. 5B) World map with approximate locations of A. boonei field samples. Those that tested positive for the GH25 muramidase domain are indicated by green stars and those without are indicated by red stars. Map is a public domain image from Wikimedia Commons.

FIG. 6. PCR amplifications testing genomic integration with primers within and outside of lysozyme genes. Primers used are listed in Table 3 and binding sites are indicated in gene diagrams with small black arrows. All integrations were confirmed with Sanger sequencing. Abbreviations: Sm: S. moellendorffii, L: Lao Spreading Center, -: water only control, CHP=conserved hypothetical protein, App-1=ADP-ribose-1″monophosphatase, PRT=phosphoribosyltransferase.

FIG. 7. Lysozyme purifications. PAGE gel stained with GelCode blue before and after purification of 6×-histidine tagged enzymes using nickel affinity chromatography. L=crude E. coli lysate expressing the indicated lysozyme, E=elution after lysozyme purification. P. poly=P. polymyxa lysozyme, PhiBP=bacteriophage PhiBP lysozyme, A. boo=A. boonei GH25 domain.

FIG. 8. Antibacterial test of A. boonei GH25 muramidase on non-Firmicutes bacteria. Bacteria of the specified strain/species incubated overnight on tryptic soy agar after a 20-minute liquid preincubation with the proteins indicated. Genera: L=Listeria, S=Staphylococcus, E. saccharolyticus=Enterococcus, M=Micrococcus, E. cloacae=Enterobacter, E. coli=Escherichia, S=Serratia, D=Deinococcus. Proteins: CEWL=chicken egg white lysozyme, P. poly=P. polymyxa lysozyme, PhiBP=bacteriophage PhiBP lysozyme, A. boo=GH25 domain of A. boonei lysozyme, CFP=cyan fluorescent protein. Images are representative of at least three independent experiments.

FIGS. 9A-B. E. coli death following full length A. boonei lysozyme expression. (FIG. 9A) Live/dead stain of BL21 (DE3) E. coli transformed with expression constructs for the full-length lysozyme from A. boonei or a control lysozyme, after overnight growth without induction. PAGE gels of crude E. coli lysates from E. coli expressing the indicated lysozyme after 6 hr of induction are also shown with the expected sizes of lysozymes indicated with arrows. (FIG. 9B) Structure of original full-length A. boonei lysozyme expression plasmid and two spontaneous knockout mutants caused by insertion of IS1 transposase sequences. Knockout mutants grew to normal colony size, while all wild type colonies had intact expression plasmids, grew poorly, and died over time in liquid culture.

FIGS. 10A-D. Lysozyme expression and relative fitness during A. boonei and M. lauensis coculture. (FIG. 10A) Expression of A. boonei GH25 muramidase relative to the control gene elongation factor 1α, after the indicated time of coculture with M. lauensis (M.l) at the specified ratio relative to A. boonei. * P<0.05, ** P<0.01, by Mann-Whitney U pairwise comparisons. N=6 for all samples. Primers are listed in Table 3. (FIG. 10B) Relative fitness of A. boonei vs. M. lauensis in monoculture (N=5) and coculture (N=4). (FIG. 10C) Growth of A. boonei (red) and M. lauensis (blue) monocultures over time. Significant differences in cell abundance occur at 24, 52, and 64 hours (P<0.05), and 56 and 60 hours (P<0.01) based on pairwise Wilcoxon tests. (FIG. 10D) Growth of A. boonei and M. lauensis in coculture over time. Significant differences in cell abundance occur at 48, 52, and 64 hours (P<0.05) based on pairwise Wilcoxon tests. Error bars are +/−SEM for all panels.

FIG. 11. The A. boonei lysozyme (GH25 muramidase) is antibacterial after an 85° C. heat shock for 20 minutes. Specifically, the purified GH25 muramidase inhibits Bacillus subtilis colony growth. In contrast, chicken egg white lysozyme (CEWL) is not antibacterial after heat shock and leads to similar colony growth as the phosphate buffer saline (PBS) negative control. The thermotolerance of the GH25 muramidase is of particular interest because of its relevance to industrial applications and the warm human body.

### DESCRIPTION OF ILLUSTRATIVE EMBODIMENTS

In this study, the inventors demonstrate that a functional antibacterial gene family has scattered across the tree of life in diverse ecological contexts. This bacterial gene encodes a glycosyl hydrolase 25 (GH25) muramidase, a peptidoglycan-degrading lysozyme that hydrolyze the 1,4-β-linkages between N-acetylmuramic acid and N-acetyl-D-glucosamine in the bacterial cell wall. Typically found in bacteria (Cantarel et al., 2009), the lytic enzyme functions in cell division and cell wall remodeling (Vollmer et al., 2008), while in bacteriophages they lyse host cells at the end of the phage life cycle (Fastrez, J. 1996). Although members of the GH25 muramidase family have been noted in other taxa (Korczynska et al., 2010; Nikoh et al., 2010), extensive analysis of their evolutionary history and functions have not been undertaken. The inventors hypothesized that similar to the transfer of antibiotic resistance genes between bacteria, the transfer of antibacterial genes from bacteria to archaea and eukaryotes bestows a selective advantage to nonbacterial taxa that must universally deploy antibacterial traits to survive in environments dominated by bacteria.

These and other aspects of the disclosure are described in greater detail below.

### I. GH25

### A. Structure

The GH25 muramidase in A. boonei is part of a lysozyme containing three additional conserved protein domains: one amidase 6 domain of unknown function and two SH3 domains that may be involved in protein-protein or protein-peptidoglycan binding. The GH25 domain itself is predicted to be a β-barrel protein consisting of seven parallel β-strands and one anti-parallel β-strand, flanked by at least three α-helices. GH25 muramidases contain a conserved DxE active site motif, where D is aspartate, x is any amino acid, and E is glutamate.

### B. Function

GH25 muramidases cleave the β-1-4-glycosidic bond between N-acetylglucosamine and N-acetylmuramic acid in the carbohydrate backbone of peptidoglycan. These domains are most often found in bacteria and bacteriophages, where they function in cell wall remodeling, autolysis, and bacteriophage lytic exit from the host.

### II. POLYPEPTIDE PRODUCTION

### A. Recombinant Production

In general, recombinant production will be utilized to express GH25 polypeptides. Nucleic acids encoding GH25 will be linked to other sequences to effect the expression of GH25 in host cells. Expression vectors containing all the information necessary to express the protein are used. Expression requires that appropriate signals be provided in the vectors, and which include various regulatory elements, such as enhancers/promoters that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Sambrook et al. (1989) and Ausubel et al. (1994), both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Regulatory Elements

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally-associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally-occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202, U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively controls the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

2. Multiple Cloning Sites

Vectors can include a multiple cloning site (MCS), which is a nucleic acid region that contains multiple restriction enzyme sites, any of which can be used in conjunction with standard recombinant technology to digest the vector. See Carbonelli et al., 1999, Levenson et al., 1998, and Cocea, 1997, incorporated herein by reference. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

3. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and/or to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

4. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Polyadenylation may increase the stability of the transcript or may facilitate cytoplasmic transport.

5. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

6. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

7. Viral Vectors

The capacity of certain viral vectors to efficiently infect or enter cells, to integrate into a host cell genome and stably express viral genes, have led to the development and application of a number of different viral vector systems. Viral systems are currently being developed for use as vectors for ex vivo and in vivo gene transfer. For example, adenovirus, herpes-simplex virus, retrovirus and adeno-associated virus vectors are being evaluated (Robbins and Ghivizzani, 1998; Imai et al., 1998; U.S. Pat. No. 5,670,488, incorporated herein by reference in its entirety). The various viral vectors described below, present specific advantages and disadvantages, depending on the particular gene-therapeutic application.

8. Non-Viral Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); or by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985). Through the application of techniques such as these, organelle(s), cell(s), tissue(s) or organism(s) may be stably or transiently transformed.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

9. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

An exemplified expression system is an RNA polymerase expression system that is highly selective for bacteriophage T7 RNA polymerase. The initial system involved two different methods of maintaining T7 RNA polymerase into the cell—in one method, a lambda bacteriophage was used to insert the gene which codes for T7 RNA polymerase, and in the other, the gene for T7 RNA polymerase was inserted into the host chromosome. This expression system has become known as the pET Expression System, and is now widely used because of its ability to mass-produce proteins, the specificity involved in the T7 promoter which only binds T7 RNA polymerase, and also the design of the system which allows for the easy manipulation of how much of the desired protein is expressed and when that expression occurs.

The insect cell/baculovirus system can produce a high level of protein expression of a heterologous nucleic acid segment, such as described in U.S. Pat. Nos. 5,871,986 and 4,879,236, both herein incorporated by reference, and which can be bought, for example, under the name MaxBac® 2.0 from Invitrogen® and BacPack™ Baculovirus Expression System From Clontech®.

Other examples of expression systems include Stratagene®'s Complete Control™ Inducible Mammalian Expression System, which involves a synthetic ecdysone-inducible receptor, or its pET Expression System, an E. coli expression system. Another example of an inducible expression system is available from Invitrogen®, which carries the T-Rex™ (tetracycline-regulated expression) System, an inducible mammalian expression system that uses the full-length CMV promoter. Invitrogen® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented.

One embodiment of the foregoing involves the use of gene transfer to immortalize cells for the production of proteins. The gene for the protein of interest may be transferred as described above into appropriate host cells followed by culture of cells under the appropriate conditions. The gene for virtually any polypeptide may be employed in this manner. The generation of recombinant expression vectors, and the elements included therein, are discussed above. Alternatively, the protein to be produced may be an endogenous protein normally synthesized by the cell in question.

Examples of useful mammalian host cell lines are Vero and HeLa cells and cell lines of Chinese hamster ovary, W138, BHK, COS-7, 293, HepG2, NIH3T3, RIN and MDCK cells. In addition, a host cell strain may be chosen that modulates the expression of the inserted sequences, or modifies and process the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection for dhfr that confers resistance to; gpt, that confers resistance to mycophenolic acid; neo, that confers resistance to the aminoglycoside G418; and hygro, that confers resistance to hygromycin.

Protein sequences may be produced using the solid-phase synthetic techniques (Merrifield, 1963). Other synthesis techniques are well known to those of skill in the art (Bodanszky et al., 1976; Peptide Synthesis, 1985; Solid Phase Peptide Synthelia, 1984). Appropriate protective groups for use in such syntheses will be found in the above texts, as well as in Protective Groups in Organic Chemistry, 1973. These synthetic methods involve the sequential addition of one or more amino acid residues or suitable protected amino acid residues to a growing peptide chain. Normally, either the amino or carboxyl group of the first amino acid residue is protected by a suitable, selectively removable protecting group. A different, selectively removable protecting group is utilized for amino acids containing a reactive side group, such as lysine.

Using solid phase synthesis as an example, the protected or derivatized amino acid is attached to an inert solid support through its unprotected carboxyl or amino group. The protecting group of the amino or carboxyl group is then selectively removed and the next amino acid in the sequence having the complementary (amino or carboxyl) group suitably protected is admixed and reacted with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to provide the final peptide. The peptides of the disclosure are preferably devoid of benzylated or methylbenzylated amino acids. Such protecting group moieties may be used in the course of synthesis, but they are removed before the peptides are used. Additional reactions may be necessary, as described elsewhere, to form intramolecular linkages to restrain conformation.

Aside from the twenty standard amino acids can be used, there are a vast number of “non-standard” amino acids. Two of these can be specified by the genetic code, but are rather rare in proteins. Selenocysteine is incorporated into some proteins at a UGA codon, which is normally a stop codon. Pyrrolysine is used by some methanogenic archaea in enzymes that they use to produce methane. It is coded for with the codon UAG. Examples of non-standard amino acids that are not found in proteins include lanthionine, 2-aminoisobutyric acid, dehydroalanine and the neurotransmitter gamma-aminobutyric acid. Non-standard amino acids often occur as intermediates in the metabolic pathways for standard amino acids—for example ornithine and citrulline occur in the urea cycle, part of amino acid catabolism. Non-standard amino acids are usually formed through modifications to standard amino acids. For example, homocysteine is formed through the transsulfuration pathway or by the demethylation of methionine via the intermediate metabolite S-adenosyl methionine, while hydroxyproline is made by a posttranslational modification of proline.

### C. Fusion Proteins

The GH25 proteins may advantageously be linked to other proteinaceous sequences to create “fusion” proteins that contain two protein segments not normally found together in nature. For example, one may wish to link GH25 to a protein purification domain, such as 6×His, or to a second antimicrobial peptide that could act in concert with GH25 to limit infection. Fusions may be genetic in nature, i.e., the nucleic acid may encode the entire fusion protein, or may be chemical, where two polypeptides are synthesized separately and joined via a post-translationally induced bond.

Linkers or cross-linking agents may be used to fuse GH25 polypeptides to other proteinaceous sequences. Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino-, sulfhydryl-, guanidino-, indole-, or carboxyl-specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

In another example, heterobifunctional cross-linking reagents and methods of using the cross-linking reagents are described in U.S. Pat. No. 5,889,155, specifically incorporated herein by reference in its entirety. The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides. In instances where a particular peptide does not contain a residue amenable for a given cross-linking reagent in its native sequence, conservative genetic or synthetic amino acid changes in the primary sequence can be utilized.

### IV. THERAPIES AND TREATMENTS

### A. Pharmaceutical Formulations and Routes of Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render agents stable. Buffers also will be employed when agents are introduced into a patient. Aqueous compositions of the present disclosure comprise an effective amount of the agent to target cells or patients, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the agents of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present disclosure may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### B. Infectious Disease States

The present compositions are useful in the treatment of infectious diseases. In particular, the inventors contemplate treatment of Bacillaceae and Paenibacillaceae infections, including B. subtilis, B. megaterium and Paenibacillus polymyxa. Another significant pathogen that may be treated in accordance with the present disclosure is Eschericia coli. The compositions, in particular full-length lysozyme, may also be useful for treatment of numerous human, plant crop and veterinary pathogens. Of special interest are Firmicutes pathogens such as Bacillus anthracis, Bacillus cereus, and Staphylococcus aureus.

### C. Treatment Methods

The agents of the present disclosure are intended for use as antimicriobial agents. They can be administered to mammalian subjects (e.g., human patients) alone or in conjunction with other antibacterial or antimicrobial drugs. The compounds can also be administered to subjects that are infected with microbes, are prone to microbial infection, or will be exposed to an environment where exposure to such microbes is likely.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.0001-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more times). Encapsulation of the polypeptide in a suitable delivery vehicle (e.g., polymeric microparticles or implantable devices) may increase the efficiency of delivery, particularly for oral delivery.

### D. Combination Therapies

It is common in many fields of medicine to treat a disease with multiple therapeutic modalities, often called “combination therapies.” To treat infectious disease using the methods and compositions of the present disclosure, one would generally contact a target cell or subject with a GH25 domain polypeptide and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the GH25 polypeptide and the other includes the other agent.

Alternatively, the GH25 polypeptide may precede or follow the other treatment by intervals ranging from minutes to weeks. One would generally ensure that a significant period of time did not expire between the time of each delivery, such that the therapies would still be able to exert an advantageously combined effect on the target cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the GH25 domain polypeptide or the other therapy will be desired. Various combinations may be employed, where the GH25 domain polypeptide is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated.

Agents or factors suitable for use in a combined therapy against an infectious disease include antibiotics such as penicillins, cephalosporins, carbapenems, macrolides, aminoglycosides, quinolones (including fluoroquinolones), sulfonamides and tetracyclines.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating infections.

### E. Non-Medicinal Treatments

There are numerous non-medicine treatments that may employ the muramidase GH25 domain. For example, in agriculture, the muramidase GH25 domain may be used to treat growing or harvested produce. It may also be used in secondary food production, such as in the dairy or meat industry. It may be used in toothpastes, eye drops or skin creams. It can also be used as a general antiseptic or cleaning agent. Below are some non-limiting examples.

Biofilm Treatment.

Microorganisms growing in biofilms are less susceptible to all types of antimicrobial agents than the same microorganisms when grown in conventional suspension cultures, and the muramidase GH25 domain can be used in this setting. It is well known that starved bacteria can be much less susceptible to a variety of antimicrobial challenges. For example, a number of classical antibiotics such as penicillin, perform poorly in slow or non dividing bacteria. In particular, biofilm control in dental water lines can be a major problem. Biofilm buildup within a dental water line can contain biofilms consisting of Psuedomonas aeroginosa, Proteus mirabilis, and Leigonella sp. to name but a few. There is also the possibility of colonisation of species generally found within the oral cavity as a result of the failure of anti retraction valves within the system. The risk of cross infection becomes even more of a potential risk of course when immuno-compromised patients are involved. The need exists for effective control of bacterial biofilm accumulation in dental water lines.

Toothpaste.

The muramidase GH25 domain can be used alone or in combination with other enzymes or even antimicrobial peptides. A typical toothpaste composition including the muramidase GH25 domain would include inactive ingredients such as Glucose Oxidase, Lysozyme, Sodium Monofluorophosphate, Sorbitol, Glycerin, Calcium Pyrophosphate, Hydrated Silica, Zylitol, Cellulose Gum, Flavor, Sodium Benzoate, Beta-d-glucose, Potassium Thiocyanate

Detergent Composition.

The muramidase GH25 domain may be added to and thus become a component of a detergent composition, particularly in a liquid detergent having a pH of 7 or lower. The detergent composition of the invention may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations. In a specific aspect, the invention provides a detergent additive comprising the muramidase GH25 domain. The detergent additive as well as the detergent composition may comprise one or more other enzymes such as a protease, a lipase, a cutinase, an amylase, a carbohydrase, a cellulase, a pectinase, a mannanase, an arabinase, a galactanase, a xylanase, an oxidase, e.g., a laccase, and/or a peroxidase. In general the properties of the chosen enzyme(s) should be compatible with the selected detergent, (i.e., pH-optimum, compatibility with other enzymatic and non-enzymatic ingredients, etc.), and the enzyme(s) should be present in effective amounts.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Reagents:

Unless otherwise stated, reagents were obtained from Fisher Scientific (Waltham, Wis.).

PCR and Sequencing:

PCR was performed using GoTaq DNA Polymerase (Promega, Madison, Wis.) with primers listed in Table 3. PCR products were electrophoresed using 1% agarose gels in sodium boric acid buffer. Following electrophoresis, gels were dyed with GelRed (Phenix Research, Candler, N.C.) and imaged on an Alpha Innotech GelRed Imager (Alpha Innotech, San Leandro, Calif.). Amplified bands were excised from the gels and purified with an SV Wizard Gel Cleanup kit (Promega). Following purification, DNA concentration was measured using the Qubit DNA high sensitivity kit (Life Technologies, Grand Island, N.Y.) and sequencing reactions were performed by Genewiz (South Plainfield, N.J.).

Bioinformatics:

The lysozyme protein from Wolbachia prophage WORiA (ZP_{—}00372884) was used as a query in a blastp search of the NCBI nonredundant protein database using Geneious Pro v5.5.6. All hits with E-values below 10₋₁₂ were collected and duplicate entries were removed. Sequences from field and laboratory samples were added to this collection and aligned with MUSCLE (Edgar, R C, 2004), insertions and deletions were removed, and ProtTest (Abascal et al., 2005) was used to determine the best model of protein evolution based on the corrected Akaike information criterion (AICc). MrBayes (Ronquist et al., 2012) and PhyML (Guindon et al., 2010) were used to build a phylogenetic tree with Bayesian and maximum likelihood methods, respectively. For the global lysozyme phylogeny, the best model chosen by ProtTest (LG+I+G) was used to generate the maximum likelihood tree, while the 3rd best model (WAG+I+G; ΔAICc: 74.82) was used to generate the Bayesian tree due to a lack of LG model availability in MrBayes. For the aphid lysozyme phylogeny, the best model (HIVw) was again used to generate the maximum likelihood tree, while CpREV (ΔAICc: 18.77) was used for the Bayesian tree. S. sanguinolenta and S. stauntoniana lysozymes were excluded from this analysis because frameshift mutations suggest the genes may be evolving in the absence of selection, while Aphidinae lysozymes were not included because of shorter sequences of the GH25 muramidase domain obtained through the use of degenerate primers that would have limited resolution of the tree.

To test for positive selection, three A. boonei lysozyme sequences (L1128, L781, and M641), P. polymyxa lysozyme (YP_{—}003869492), and PhiBP lysozyme (CBA18122) were analyzed with three PAML-based algorithms using Datamonkey (Delport et al., 2010). Pairwise comparisons between each A. boonei lysozyme and either P. polymyxa or PhiBP were performed using DnaSP (Libradon & Rozas, 2009) with a sliding window of 9 nucleotides and a step size of 3. Residues with dN/dS ratios above 5 in multiple pairwise comparisons and/or residues indicated as under positive selection by Datamonkey, along with the 8 most highly conserved residues from the MUSCLE alignment, were mapped to a structure prediction of A. boonei lysozyme using PyMOL. Structure prediction was performed using the homology-based modeling tool Phyre2 (Kelley & Sterberg, 2009).

Lysozyme Cloning and Purification:

A. boonei GH25 muramidase domain (ZP_{—}04874596), P. polymyxa lysozyme (YP_{—}003869492), and PhiBP lysozyme (CBA18122) were cloned and expressed with a 6×C-terminal histidine tag using an Expresso T7 Cloning and Expression System (Lucigen, Middleton, Wis.) according to the manufacturers instructions. Sequence-confirmed expression plasmids and a control plasmid expressing cyan fluorescent protein (CFP) were transformed into HI-Control BL21 (DE3) E. coli cells. Cultures at an OD₆₀₀ of ˜0.5 were induced with 1 mM IPTG for 6 hours, centrifuged, and frozen at −80° C. until purification. Frozen pellets were resuspended in lysis buffer containing 10 mM Tris-HCl, pH 7.5, 300 mM NaCl, 0.5% Triton x-100, 0.3% sodium dodecyl sulfate, and 1 mM phenylmethylsulfonylfluoride and sonicated 5 times for 30 seconds with at least 1 minute on ice between sonications. Samples were centrifuged and recombinant proteins were purified from supernatant using HisPur Ni-NTA chromatography cartridges (Thermo Scientific, Waltham, Mass.) according to manufacturer's instructions. Glycerol at a final concentration of 40% was added to enzymes in elution buffer for storage at −20° C. for a maximum of three weeks before use in antibacterial assays. Purifications were analyzed with denaturing polyacrylamide gel electrophoresis and stained with GelCode Blue (Thermo Scientific).

Full-length A. boonei lysozyme and WORiA lysozyme were cloned into a pET-20b vector (EMD Millipore, Darmstadt, Germany) with a C-terminal 6× histidine tag and sequence-confirmed plasmids were transformed into BL21 (DE3) E. coli (EMD Millipore). Three colonies from each transformation were inoculated into LB media and grown to an OD600 of ˜0.5, induced for 4 hours with 1 mM IPTG and harvested for analysis on PAGE gels. Overnight cultures without induction were examined for bacterial death with a BacLight Live/Dead Stain (Life Technologies).

Antibacterial Assays:

Purified A. boonei GH25 muramidase, P. polymyxa lysozyme, PhiBP lysozyme, CFP, and commercially purchased CEWL (Sigma-Aldrich, St. Louis, Mo.) were diluted to 100 μg/mL in buffer EG (60% nickel column elution buffer, 40% glycerol) and filter sterilized. Bacteria to be tested were grown overnight in tryptic soy broth, split 1:10, and incubated to exponential growth before being diluted into each enzyme solution. Samples were incubated with shaking for 20 minutes at 37° C. and then 5 μL was spotted onto tryptic soy agar and incubated overnight at 37° C. To evaluate whether antibacterial activity is dose-dependent, B. subtilis was incubated with A. boonei GH25 muramidase at 100 μg/mL, 75 μg/mL, 50 μg/mL, 25 μg/mL and 0 μg/mL and 100 μl was spread on tryptic soy agar plates. Replicates of 10 were performed for each concentration, plates were incubated overnight at 37° C., and colonies were counted the following morning. Bacterial strains used in these experiments are listed in Extended Data Table 2.

A. boonei Cultures:

A. boonei and M. lauensis cultures were performed as previously described (Reysenbach et al., 2006) with the following modifications: yeast extract was added at 2.0 g/L, pH was adjusted to 4.8, and cultures were incubated at 65° C. For gene expression studies, 8.2×10⁵ cells were inoculated into 5 mL cultures in 6 replicates each of monocultures and cocultures at 0.1:1, 1:1, and 1:0.1 ratios and 500 μL samples were collected after 4 and 12 hours of co-incubation and frozen for expression analysis. RNA was isolated from frozen samples using an RNeasy Mini Kit (Qiagen) and QIAshredder (Qiagen), DNA contamination was removed with a Turbo DNAfree Kit (Life Technologies), and reverse transcription was performed using a Superscript III 1ₛₜ Strand Synthesis System (Life Technologies) along with no-reverse transcriptase controls. Quantitative PCR was performed with GoTaq qPCR Master Mix (Promega) using a CFX96 Real-Time System (Bio-Rad, Hercules, Calif.). Primers are listed in Table 3. For competition studies, 5 replicates of 5 mL cultures were inoculated as monocultures or 1:1 cocultures and 175 μL was collected every 4 hours for counting of relative species abundance with a hemocytometer. Relative fitness was calculated based on Malthusian parameters over the period of exponential growth as previously described (Reysenbach et al., 2013).

### Example 2

### Results and Discussion

GH25 Muramidases are Present in Non-Bacterial Species:

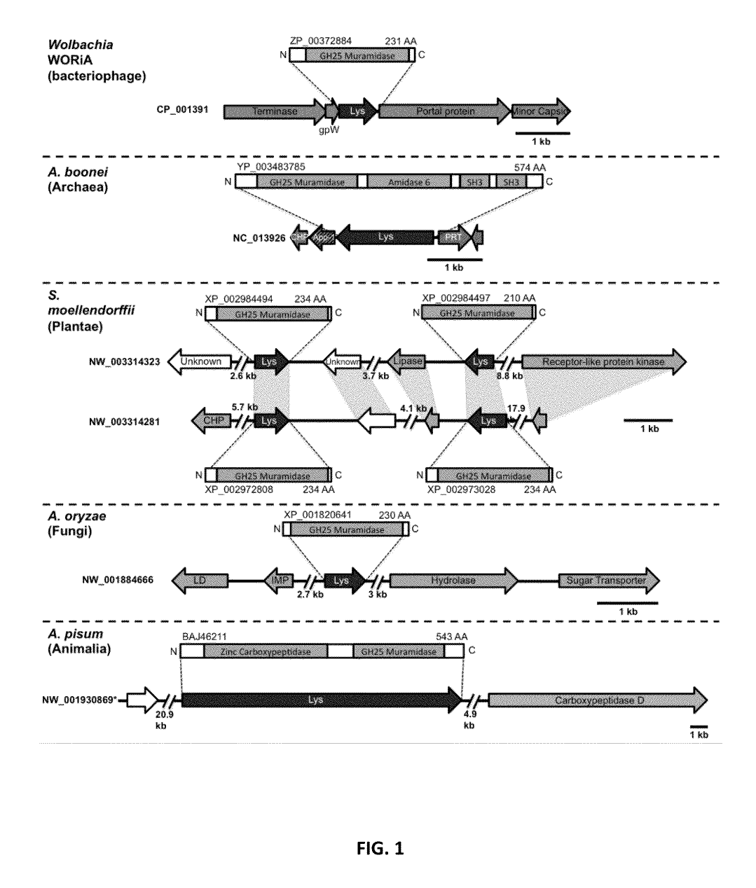

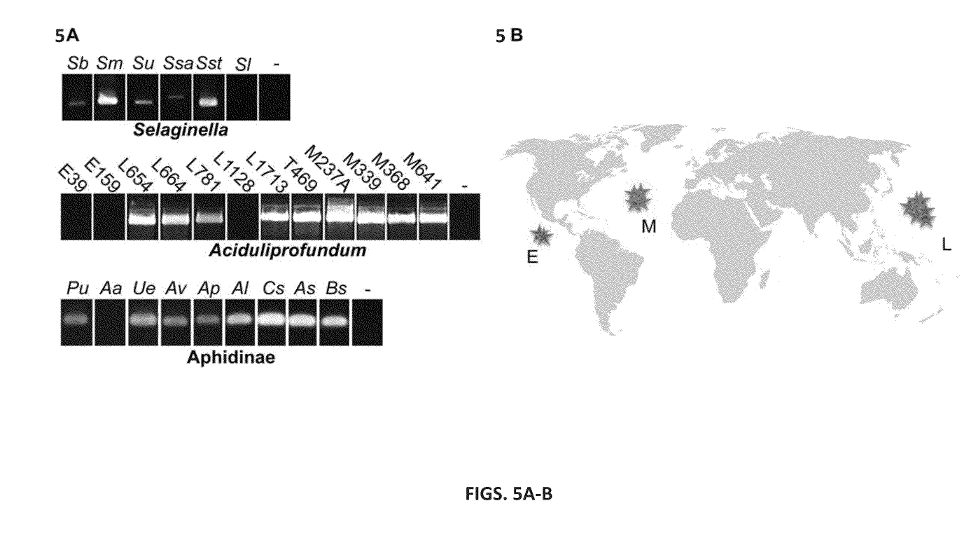

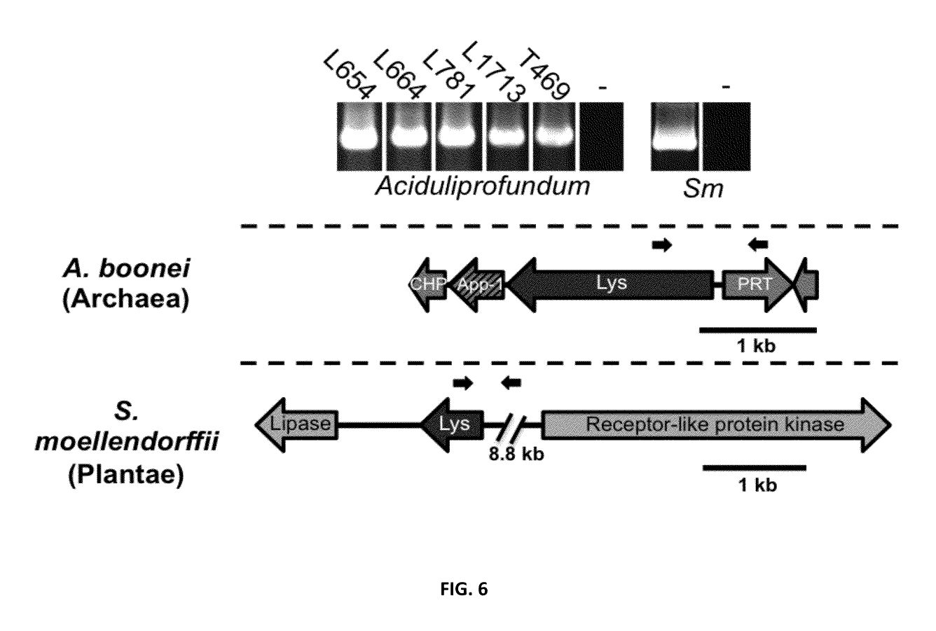

During a homology search, the inventors uncovered 75 nonredundant homologs (E-values≦10⁻¹²) of a bacterial GH25 muramidase in disparate taxa across the tree of life, indicating possible HGT of a bacterial gene to both eukaryotic and archaeal species as well as phages. Putative HGT events were identified in the genomes of the plant Selaginella moellendorffii (Banks et al., 2011), the deep-sea hydrothermal vent archaeon Aciduliprofundum boonei (Reysenbach et al., 2006), the pea aphid Acyrthosiphon pisum (Nikoh et al., 2010; Richards et al., 2010), and several species of fungi such as Aspergillus oryzae (Machida et al., 2005). To rule out spurious bacterial contamination in these genomes, the inventors verified the presence of the lysozyme gene in natural populations of selected HGT recipients by PCR and sequencing of the GH25 muramidase domain (FIG. 5), including Aciduliprofundum field samples harvested from hydrothermal vents worldwide. The inventors detected lysozyme genes in 9 out of 12 field isolates of Aciduliprofundum from deep-sea vents in the Atlantic and Pacific oceans, 5 out of 6 species in the plant genus Selaginella, and 8 out of 9 aphid species in the subfamily Aphidinae (Table 1). The inventors also found lysozymes in two additional WO phages as part of an ongoing next generation sequencing project of Wolbachia viruses (unpublished data). Examination of the genomic surroundings of these HGT recipients revealed non-bacterial flanking genes on either side of the transferred lysozyme in each case (FIG. 1). To confirm genomic integration, the inventors employed PCR and sequencing on a subset of these samples using primers within and outside of the lysozyme gene. Incorporation of the lysozyme gene was verified in all cases tested (FIG. 6).

Non-Bacterial GH25 Muramidases are the Product of HGT:

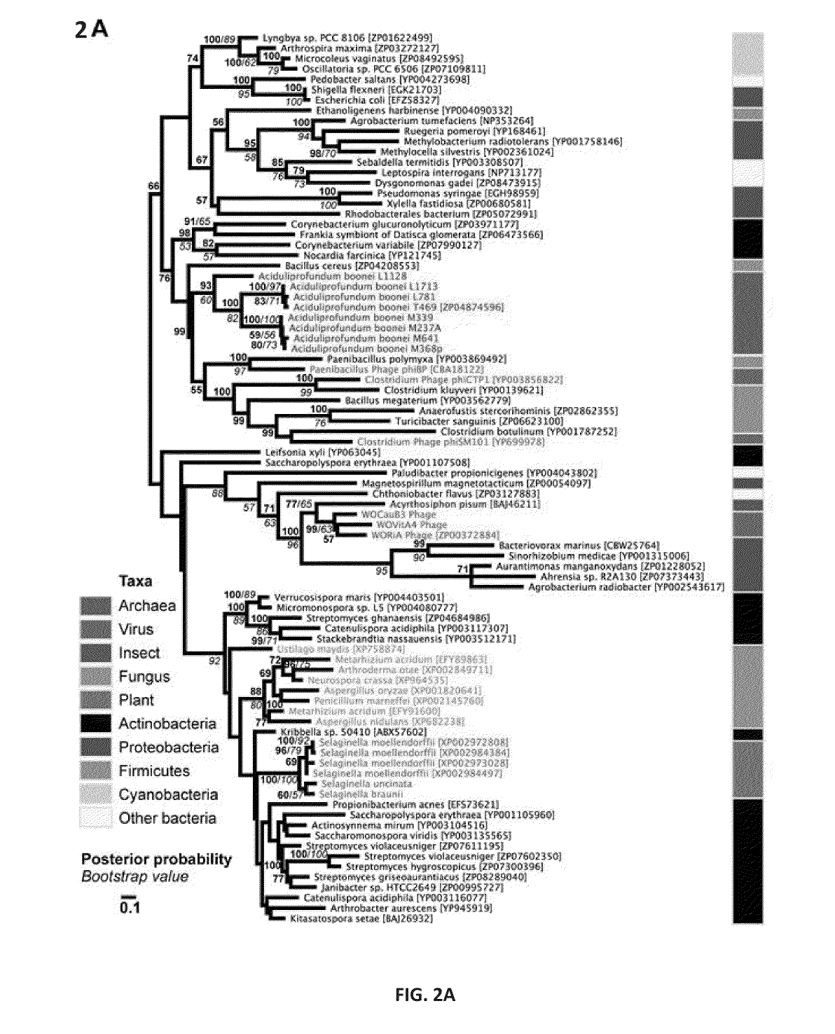

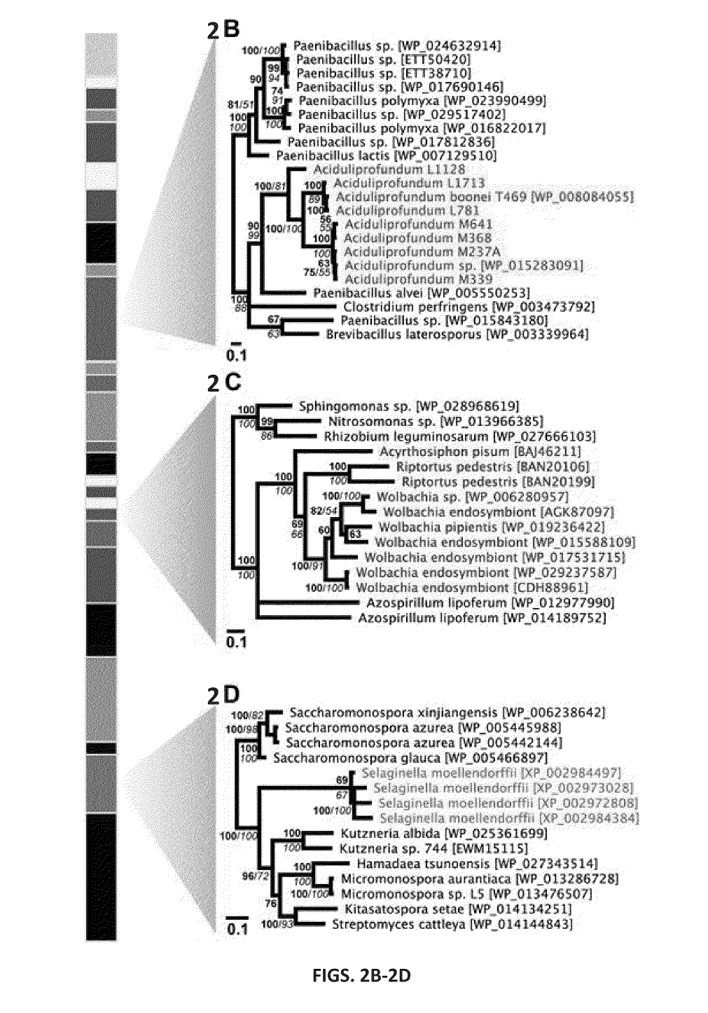

To further establish HGT, the inventors conducted a phylogenetic analysis on 86 GH25 muramidase sequences using Bayesian and maximum likelihood inference methods (FIG. 2). The inventors combined non-redundant Aciduliprofundum, Selaginella, and WO sequences obtained from PCR and Sanger sequencing with blastp results to reconstruct the phylogeny. Three key results emerge from the phylogenetic analysis: (i) at least four instances of interdomain HGT of the bacterial GH25 muramidase occurred in nonbacterial species as well as a number of transfers to bacteriophages, (ii) vertical transmission of the transferred gene ensues in some descendant taxa (i.e., Aciduliprofundum and Selaginella), and (iii) frequent HGT of the muramidase between bacterial clades accompanies the interdomain transfer, indicating that transfer across the tree of life is the norm for this “spreader” gene family.

The inventors observed that each putative interdomain HGT event occurred between taxa that likely encounter each other in the same ecological niche. For instance, the A. boonei lysozyme is in a Glade dominated by Firmicutes whose members can be common in deep ocean sediments (Orcutt et al., 2011), and the S. moellendorffii lysozyme is closely related to Actinobacteria, which are dominant microbes in soil (Bulgarelli et al., 2013). The inventors found no evidence of a GH25 muramidase in >200 sequenced archaeal genomes and more than 70 plant genomes, beyond those presented in this study. Thus, the lysozyme was not in the last common ancestor of all domains, as it would require the unlikely loss of the gene in dozens of lineages while maintaining it in an exceedingly small number of species. In summary, the presence of a GH25 muramidase in nonbacterial species represents a series of recurrent, independent horizontal gene transfer events derived from diverse, ecologically associated bacteria.

Non-Bacterial GH25 Muramidases have Conserved Active Sites:

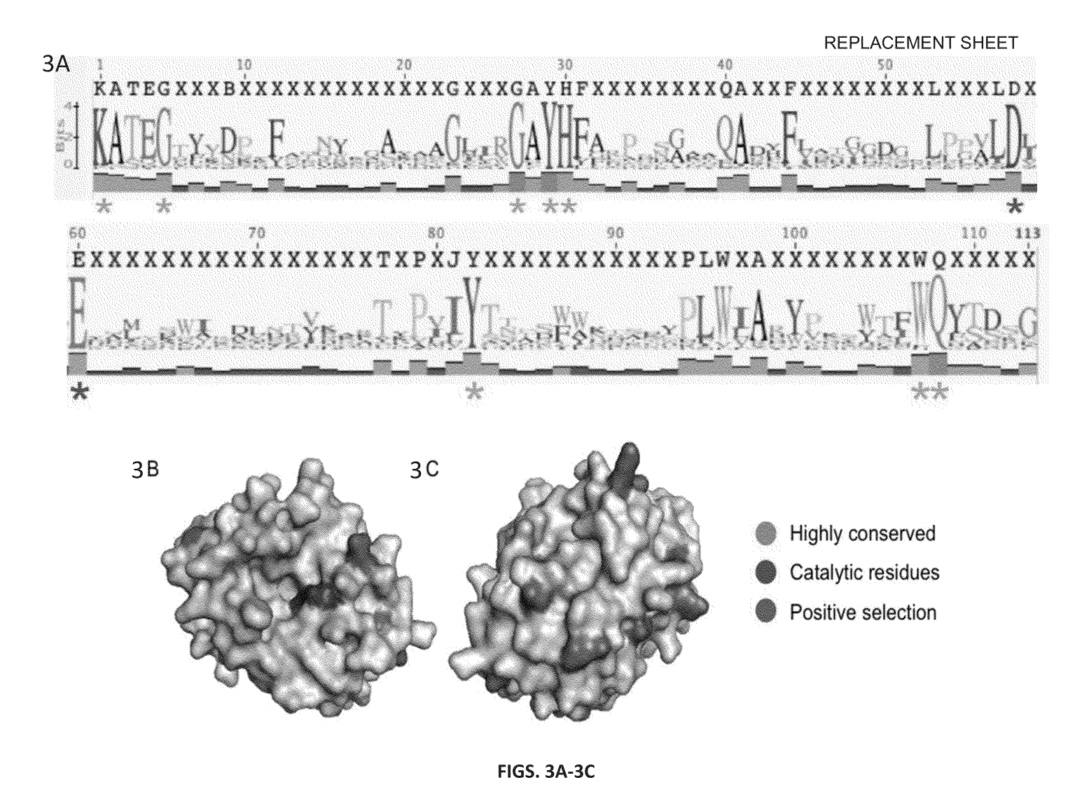

The inventors next undertook a series of experiments to test the hypothesis that the presence of the transferred muramidase functions to kill bacteria. Since HGT frequently results in pseudogenized and nonfunctional genes (Dunning et al., 2007; Nikoh et al., 2010; Kondrashov et al., 2006; Nikoh et al., 2008), they first investigated the amino acid sequences for preserved antibacterial action of the transferred lysozymes in nonbacterial genomes. They aligned all 86 GH25 muramidase sequences to identify conserved sites (FIG. 3A). They then mapped the conserved amino acids (and positively selected residues, described below) to a three-dimensional structure prediction of the A. boonei GH25 muramidase domain (FIGS. 3B-C). Highly conserved residues (>85% identity between all taxa) invariably mapped to the previously identified active site pocket (Martinez-Fleites et al., 2009). This was also true for structure predictions of other GH25 muramidases in the phylogeny such as S. moellendorffii and WORiA. The inventors next tested for positive selection between A. boonei GH25 muramidases and their most closely related homologs from the bacterium Paenibacillus polymyxa and phage PhiBP using PAML and pairwise sliding window analyses of synonymous and nonsynonymous mutations. Most amino acids were under purifying selection, but those under positive selection were among the protein's exterior residues, suggesting that external amino acids may be evolving to facilitate protein-protein or protein-peptidoglycan interactions while the active site pocket remains conserved.

A. boonei GH25 Muramidase is Antibacterial:

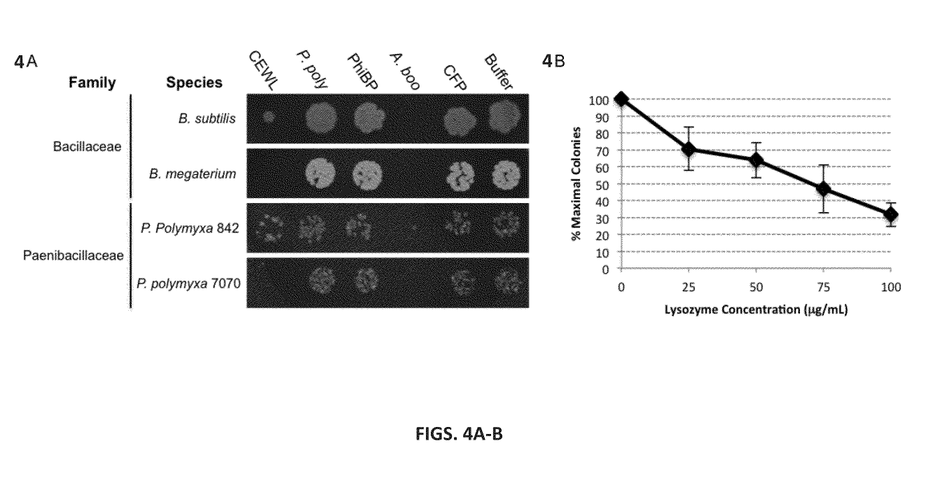

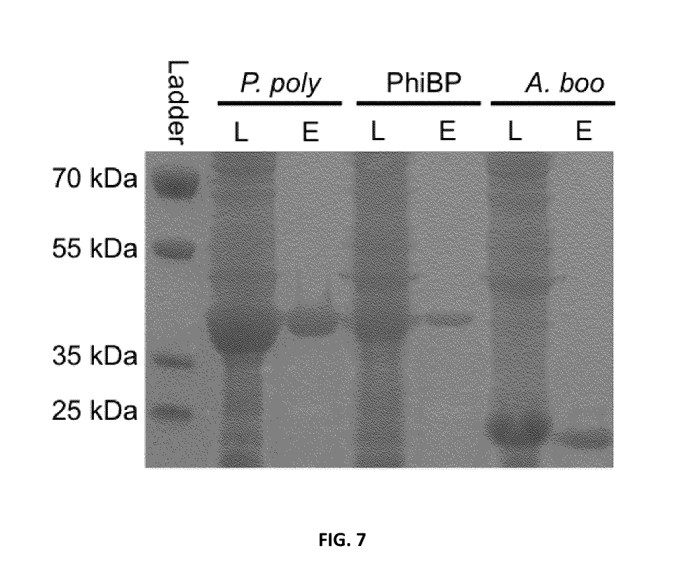

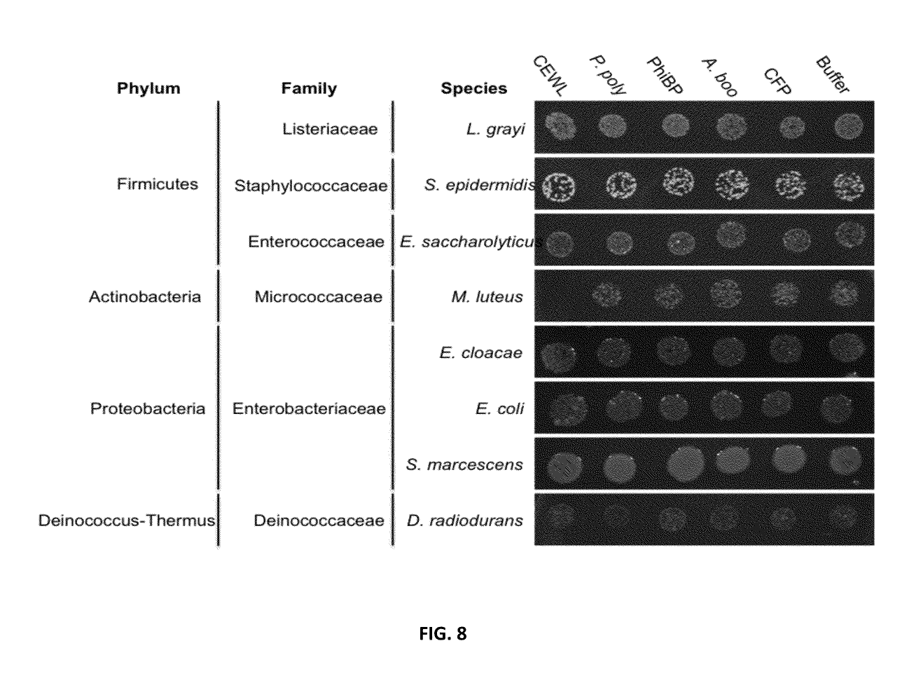

To further assess the function of the transferred lysozymes, the inventors cloned, expressed, and purified the GH25 muramidase domain from A. boonei and compared its lytic activity to that of closely related homologs in P. polymyxa and PhiBP. They obtained each muramidase in a pure elution (FIG. 7) and tested for antibacterial action against a range of bacterial species. As predicted, A. boonei GH25 muramidase efficiently killed several species of bacteria in the phylum Firmicutes—the putative donor group of the gene (FIG. 4A). The bacterial inhibition by A. boonei GH25 muramidase was more potent than the positive control, chicken egg white lysozyme, and was dose-dependent (FIG. 4B). Bacterial and phage muramidases did not elicit antibacterial killing, similar to cyan fluorescent protein and buffer-only negative controls, likely because bacteria typically use a large protein complex to limit their lysozymes' activity to the septum during cell division (Uehara and Bernhardt, 2011), and PhiBP phage has a documented spectrum of activity limited only to a P. polymyxa strain unavailable for analyses (Halgasova et al., 2010). As expected, the A. boonei GH25 muramidase did not exhibit antibacterial activity against Gram-negative species or Gram-positive species outside of the families Bacillaceae and Paenibacillaceae, which was equivalent to the killing range of chicken egg white lysozyme with the exception of the Actinobacterium M. luteus (FIG. 8).

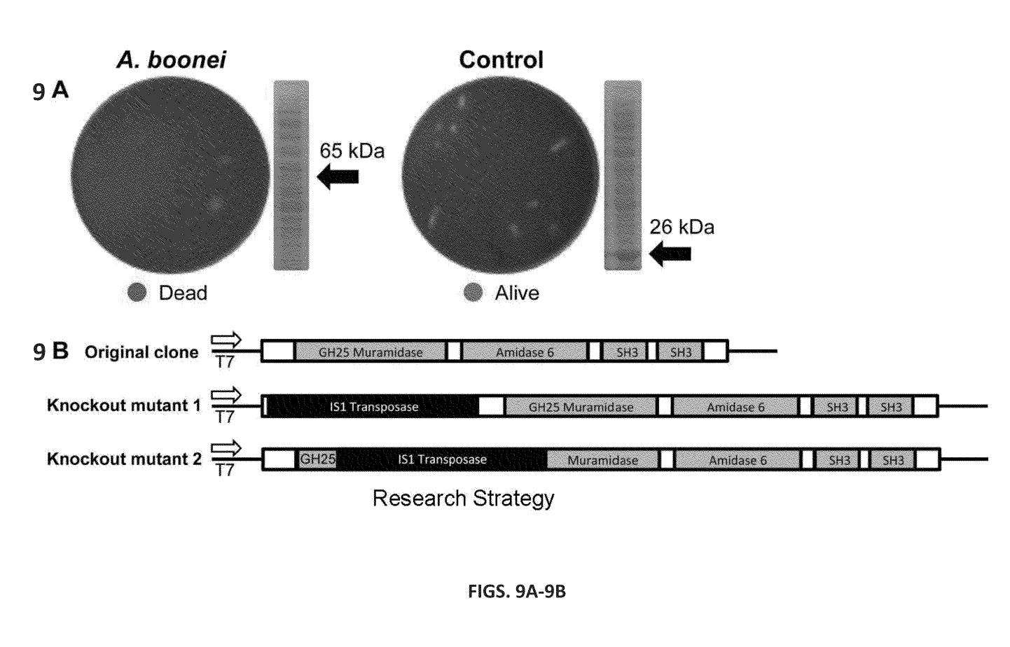

The A. boonei muramidase domain is part of a larger gene (1725 bp) composed of other domains that may broaden or constrain the range of antibacterial activity. To test its function, the inventors cloned the entire gene into an expression plasmid in E. coli and discovered that bacterial colonies grew poorly, with tiny, slow-growing colonies on solid media, and substantial cell death coinciding with a small amount of leaky expression in liquid culture. Unexpectedly, a few E. coli clones grew to normal colony size. Upon sequencing the plasmids of these thriving colonies, the inventors found frameshift mutations scrambling most of the lysozyme gene sequence (FIGS. 9A-B). Thus, expression of the complete lysozyme resulted in E. coli death, while mutated lysozyme did not, providing additional evidence that the HGT-derived lysozyme in A. boonei possesses antibacterial action against bacterial taxa.

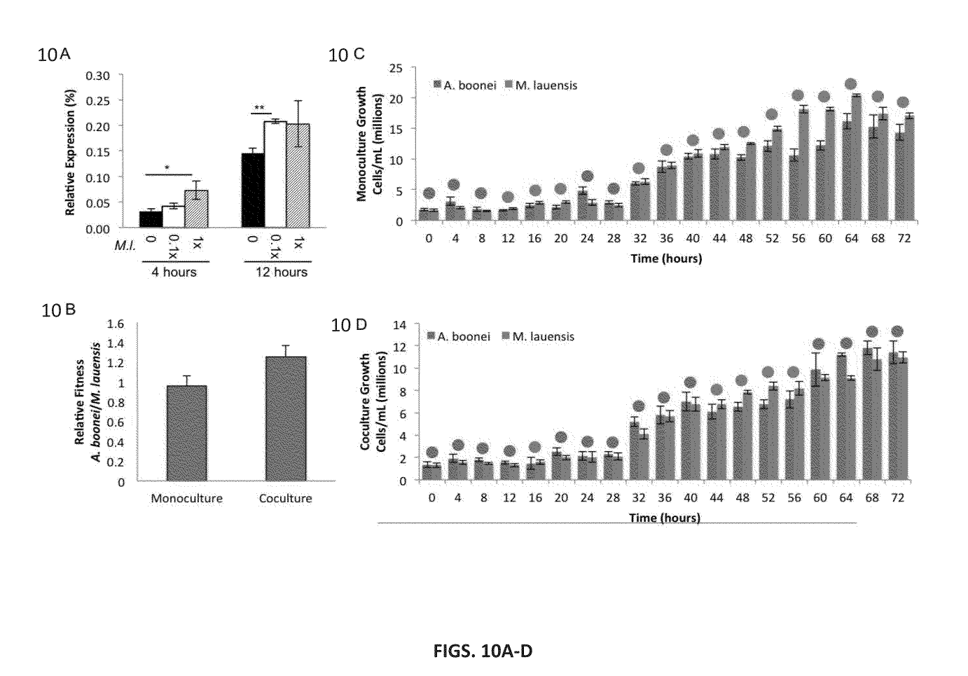

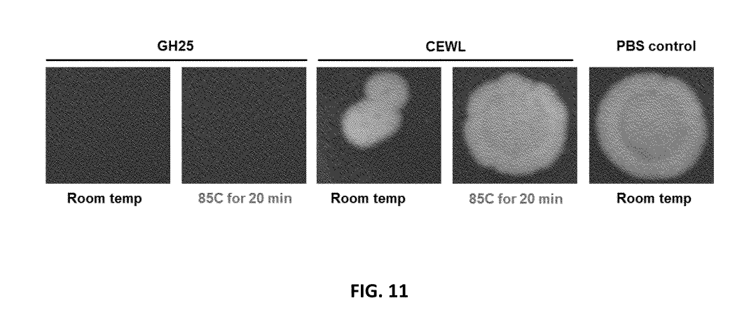

Finally, on the basis of the antibacterial killing data and the known role of lysozymes in phagemediated bacterial lysis and eukaryotic immune defense, the inventors postulated that horizontally transferred lysozymes serve as antibacterials to fend off bacterial niche competitors. Since Firmicutes are often an abundant constituent of vent microbiota in culture-independent surveys (Zhou et al., 2011; Wei et al., 2013), an ideal experiment to address this hypothesis would be to coculture a vent Firmicutes with A. boonei wild-type and lysozyme knock outs and test relative fitness and bacterial inhibition. However, such cultivable Firmicutes species are not available and genetic manipulation of A. boonei has not been accomplished. Alternatively, the inventors cultured A. boonei cells in anaerobic marine media (Reysenbach et al., 2006) with and without Mesoaciditoga lauensis from the phylum Thermotogae that co-inhabits the same deep-sea vent niche as Aciduliprofundum (Reysenbach et al., 2013). They observed a significant increase in A. boonei lysozyme expression at four and twelve hours of coculture with M. lauensis (FIG. 10A), demonstrating that A. boonei responds to the presence of this bacterial competitor by increasing production of the antibacterial lysozyme. Moreover, FIG. 10D shows the relative Malthusian fitness (Lenski et al., 1991) increase for A. boonei in coculture vs. monoculture scaled across the time period of exponential growth. This difference is marginally non-significant, perhaps due to low sample sizes (P=0.11, N=5, MWU two-tailed test).

When the species are cultured separately for 72 hours, M. lauensis cell abundance is greater than that of A. boonei during 14 out of the 19 sampling points (FIG. 10C, blue circles), indicating that bacteria outperform archaea in monoculture conditions. However, when the two species are cocultured, the cell abundances reverse and A. boonei outperforms M. lauensis for 14 out of the 19 time points (FIG. 10D, red circles). This competitive frequency difference is significant (Chi-square test, p=0.0035), complementing the Malthusian fitness increase showing that A. boonei outcompetes its bacterial competitor in coculture despite a higher monoculture growth rate for the bacteria. For each monoculture time point, there are 4.43% fewer A. boonei cells on average than M. lauensis, while in coculture there are 6.22% more A. boonei cells per time point (Mann Whitney U. p=0.023), also demonstrating a significant competitive advantage of A. boonei in the coculture experiment that associates with increased lysozyme expression, as mentioned above.

In addition, the inventors have determined that A. boonei lysozyme (GH25 muramidase) is antibacterial after an 85° C. heat shock for 20 minutes. The thermotolerance of the muramidase is of particular interest because of its relevance to pharmaceuticals in warm-blooded animal, and in certain industrial applications. In contrast, a control protein of chicken egg white lysozyme loses most of its antibacterial activity at this temperature shock.

The GH25 muramidase's current efficacy is greatly improved by the presence of an imidazole that is used in the elution buffer. When one exchanges the lysozyme from the original elution buffer (PBS+imidazole) to PBS alone, the lysozyme loses substantial antibacterial activity. Moreover, the original elution buffer without lysozyme is marginally antibacterial, suggesting the imidazole is an adjuvant of the lysozyme's antibacterial activity.

Conclusions:

Overall, these results indicate a new way in which horizontally transferred “spreader” genes with broad ecological relevance can be selected for across life's diverse lineages. A striking feature of this muramidase is that it has no nonbacterial homologs except for the taxa that it transferred into and spread within. Moreover, the recipient taxa of the muramidase have clear ecological associations with the potential donor groups of the bacteria. Since HGTs experience a gradient of decreasing frequency from within domain>between two domains>between all domains of life, evolutionarily recent and parallel gene transfer between extant groups of life may be exceptionally restricted to genes that overcome a significant valley in the fitness landscape. With the repeated gene transfer and modulation of antibacterial repertories, this one antibacterial gene family represents a prototype for parallel HGT in which the evolutionary advantages of a gene family trumps the resilient selective barriers against HGT to multiple domains. Interestingly, it has been reported that the horizontally transferred lysozyme in A. pisum exhibits differential tissue expression (Nikoh et al., 2010), and a transferred lysozyme from the fungus Aspergillus nidulans (AN6470.2) has lytic activity against Micrococcus cells (Bauer et al., 2006), providing additional evidence that these horizontally transferred genes are transcriptionally and enzymatically active.

The muramidase in a thermophilic archaea is of special note as this domain of life does not possess murein cell walls (Albers & Meyer, 2011), and genes encoding an antibacterial peptide have never before been reported (Cantarel et al., 2009). Such an enzyme that differs from, and supplements, the organism's antibacterial repertoire may confer a selective advantage to the HGT recipient over more vulnerable relatives. Indeed, members of the genus Aciduliprofundum are widespread thermoacidophiles in deep-sea hydrothermal vent chimney biofilms (Flores et al., 2012) in which bacteria are frequent inhabitants (Orcutt et al., 2011; Miroshnichenko & Bonch-Osmolovskaya, 2006). It is also possible that since Aciduliprofundum strains metabolize peptides, the lysozyme enables a nutritive strategy for scavenging resources for the archaeon. Based on this work, the inventors suspect that systematic surveys of antibacterial peptides from archaea will likely uncover a broad range of antibacterial activities (Atanasova et al., 2013; Shand K J, 2008), and may eventually offer novel therapeutics. In summary, they conclude that the evolutionary path to this unique parallel HGT was paved by the universal drive for nonbacterial taxa to compete in a bacterial world. They predict that many parallel HGT genes, once discovered, will serve antibacterial functions.

TABLE 1
Field Samples Tested for Presence of Lysozyme Gene

| Taxon | Isolate, strain, or species | Origin/Distribution |
|---|---|---|
| Aciduliprofundum | Lau09-654 | Eastern Lau Spreading |
| | Lau09-664 | Center deep-sea vents |
| | Lau09-781 | |
| | Lau09-1713 | |
| | A. boonei-T469 | |
| | Lau09-1128 | |
| | Mar08-237A | Mid-Atlantic Ridge |
| | Mar08-339 | deep-sea vents |
| | Mar08-368 | |
| | Mar08-641 | |
| | Epr07-39 | East Pacific Rise deep- |
| | Epr07-159 | sea vents |
| Selaginella | S. moellendorffii | China |
| | S. braunii | China |
| | S. uncinata | China |
| | S. lepidophylla | North America |
| | S. sanguinolenta | Japan |
| | S. stauntoniana | China |
| Aphidinae | Acyrthosiphon pisum | Worldwide |
| | Pleotrichophorus utensis | United States |
| | Artemisaphis artemisicola | North America |
| | Uroleucon erigeronensis | North America |
| | Aphis varians | North America |
| | Aphis lupini | United States |
| | Cedoaphis sp. | North America |
| | Aphthargelia symphoricarpi | North America |
| | Braggia sp. | United States |
| WO | WORiA | Drosophila simulans |
| | WOC auB3 | Cadra cautella |
| | WOV itA4 | Nasonia vitripennis |

TABLE 2
Bacterial Strain used in Antibacterial Assays

| Species/strain | Source |
|---|---|
| Bacillus megaterium | Ward's Scientific |
| Bacillus subtilis ATCC 19659 | Microbiologics, Inc. |
| Paenibacillus polymyxa ATCC 842 | Microbiologics, Inc. |
| Paenibacillus polymyxa ATCC 7070 | Microbiologics, Inc. |
| Listeria grayi ATCC 25401 | Microbiologics, Inc. |
| Staphylococcus epidermidis ATCC 49134 | Microbiologics, Inc. |
| Enterococcus saccharolyticus ATCC 43076 | Microbiologics, Inc. |
| Micrococcus luteus ATCC 49732 | Microbiologics, Inc. |
| Enterobacter cloacae | Ward's Scientific |
| Escherichia coli | Ward's Scientific |
| Serratia marcescens | Ward's Scientific |
| Deinococcus radiodurans ATCC 13939 | Microbiologics, Inc. |

American Type Culture Collection (ATCC) reference strain is indicated when available.

TABLE 3
Primers

| Target | Forward Primer (5′-3′) | Reverse Primer (5′-3′) |
|---|---|---|
| S. moellendorffii | ATGGACGTAAGTAGCTACCAAGG | TCAGCCTTTGGCGAGCTTC |
| GH25 muramidase | (SEQ ID NO: 6) | (SEQ ID NO: 7) |
| Aciduliprofundum | ATGTKTCCCACTGGCAGG | CCACCCTGTCATCGTAGAAGA |
| GH25 muramidase, | (SEQ ID NO: 8) | (SEQ ID NO: 9) |
| degenerate | | |
| Aphid GH25 | CTYTGGGGAGCATAYCATTTTGG | TTTTWCCATCKGTRTAYTGCCATAA |
| muramidase, | (SEQ ID NO: 10) | (SEQ ID NO: 11) |
| degenerate | | |
| Aciduliprofundum | GGGTGCCTCTCCTCCAATCCCC | CCACTCACCCCCGATACATTTCC |
| GH25 muramidase | (SEQ ID NO: 12) | (SEQ ID NO: 13) |
| integration | | |
| S. moellendorffii | ATGGCGTTTCATTGCTTGATCTTT | GTTGTAACATTTTTGCGCTGGAGTA |
| GH25 muramidase | (SEQ ID NO: 14) | (SEQ ID NO: 15) |
| integration | | |
| A. boonei GH25 | TCCCACTGGCAGGGAAATGTGAA | ATCCTGATGCGTGTGCCTTCTCCA |
| muramidase, qPCR | CT (SEQ ID NO: 16) | (SEQ ID NO: 17) |
| A. boonei | TGTTCATCGGCCATGTTGACCACG | GCTCTTTCCGAGTTTCTCTGCCTCCT |
| elongation factor | (SEQ ID NO: 18) | (SEQ ID NO: 19) |
| 1α, qPCR | | |

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference:
U.S. Pat. No. 5,670,155
U.S. Pat. No. 5,889,155
Bodanszky et al., J. Antibiot., 29(5):549-53, 1976.
Carbonelli et al., FEMS Microbiol. Lett. 177(1):75-82, 1999.
Levenson et al., Human Gene Ther. 9(8):1233-6, 1998.
Cocea, Biotechniques, 23(5):814-6, 1997.
Chen & Greene, Mol. Cell. Biol. 5:392-401, 2004.
Chen & Okyama, Mol. Cell. Biol. 7(8):2745-52, 1987.
Merrifield, J. Am. Chem. Soc., 85:2149-2154, 1963.
Peptide Synthesis, 1985
Protective Groups in Organic Chemistry, 1973
Remington's Pharmaceutical Sciences, 15^{th} Ed., 624-652, 1035-1038 and 1570-1580, 1990.
Solid Phase Peptide Synthelia, 1984
Popa O, Dagan T (2011) Trends and barriers to lateral gene transfer in prokaryotes. Curr Opin Microbiol 14: 615-623.
Clardy et al., (2009) Curr Biol 19: R437-441.
Nelson et al., (1999) Nature 399: 323-329.
Husnik et al., (2013) Cell 153: 1567-1578.
Dunning Hotopp et al., (2007) Science 317: 1753-1756.
Wu et al., (2013) Proc Natl Acad Sci USA 110: 7748-7753.
Gladyshev et al., (2008) Science 320: 1210-1213.
Bratke K A, McLysaght A (2008) BMC Evol Biol 8: 67.
Danchin et al., (2010) Proc Natl Acad Sci USA 107: 17651-17656.
Dunning Hotopp J C (2011) Trends in Genetics 27: 157-163.
Keeling P J, Palmer J D (2008) Nat Rev Genet 9: 605-618.
Moran et al., (2012) Mol Biol Evol. 29(9):2223-30.
Lundin et al., (2010) BMC Evol Biol 10: 383.
Koonin et al., (2003) Genome Biol 4: R19.
McClure M A (2001) Curr Protein Pept Sci 2: 313-324.
McDonald et al., (2012) Mol Biol Evol 29: 51-60.
Cantarel et al., (2009) Nucleic Acids Res 37: D233-238.
Vollmer et al., (2008) FEMS Microbiol Rev 32: 259-286.
Fastrez J (1996) Phage lysozymes. In: Jolles P, editor. Lysozymes: Model Enzymes in Biochemistry and Biology: Birkhauser Verlag. pp. 35-64.
Korczynska et al., (2010) Acta Crystallogr Sect F Struct Biol Cryst Commun 66: 973-977.
Nikoh et al., (2010) PLoS Genet 6: e1000827.
Banks et al., (2011) Science 332: 960-963.
Reysenbach et al., (2006) Nature 442: 444-447.
Richards et al., (2010) PLoS Biol 8: e1000313.
Machida et al., (2005) Nature 438: 1157-1161.
Orcutt et al., (2011) ISME J 5: 692-703.
Bulgarelli et al., (2013) Annu Rev Plant Biol 64: 807-838.
Kondrashov et al., (2006) Biol Direct 1: 31.
Nikoh et al., (2008) Genome Res 18: 272-280.
Martinez-Fleites et al., (2009) Carbohydr Res 344: 1753-1757.
Uehara T, Bernhardt T G (2011) Curr Opin Microbiol 14: 698-703.
Halgasova et al., (2010) FEMS Microbiol Lett 305: 128-135.
Zhou et al., (2011) Journal of Central South University of Technology 18: 392-398.
Wei et al., (2013) Acta Oceanologica Sinica 32: 42-51.
Reysenbach et al., (2013) Int J Syst Evol Microbiol.
Lenski et al., (1991) American Naturalist 138: 1315-1341.
Bauer et al., (2006) Proc Natl Acad Sci USA 103: 11417-11422.
Albers S V, Meyer B H (2011) Nat Rev Microbiol 9: 414-426.
Flores et al., (2012) Front Microbiol 3: 47.
Orcutt et al., (2011) Microbiol Mol Biol Rev 75: 361-422.
Miroshnichenko M L, Bonch-Osmolovskaya E A (2006) Extremophiles 10: 85-96.
Atanasova et al., (2013) Microbiologyopen 2: 811-825.
Shand R F L, K. J. (2008) Archaeal Animicrobials: an Undiscovered Country. In: Blum P, editor. Archaea: New Models for Prokaryotic Biology: Caister Academic Press. pp. 233-243.
Edgar R C (2004) Nucleic Acids Res 32: 1792-1797.
Abascal et al., (2005) Bioinformatics 21: 2104-2105.
Ronquist et al., (2012) Syst Biol 61: 539-542.
Guindon et al., (2010) Syst Biol 59: 307-321.
Delport et al., (2010) Bioinformatics 26: 2455-2457.
Librado P, Rozas J (2009) Bioinformatics 25: 1451-1452.
Kelley L A, Sternberg M J (2009) Nat Protoc 4: 363-371.

1. A pharmaceutical composition comprising an Aciduliprofundum boonei glycosyl hydrolase 25 muramidase (GH25) domain disposed in a pharmaceutically acceptable diluent, carrier or excipient.

2. The pharmaceutical composition of claim 1, wherein the muramidase domain has a sequence according to SEQ ID NO: 1.

3. The pharmaceutical composition of claim 1, wherein the muramidase domain has a sequence that is 70%, 80%, or 90% homologous to the sequence according to SEQ ID NO: 1.

4. The pharmaceutical composition of claim 1, wherein the muramidase domain has a sequence that hybridizes under high stringency conditions to a sequence according to SEQ ID NO: 2.

5. The pharmaceutical composition of claim 1, wherein the muramidase domain further comprises a sequence according to SEQ ID NO: 3.

6. The pharmaceutical composition of claim 1, wherein the muramidase domain is fused to a non-Aciduliprofundum boonei sequence.

7. The pharmaceutical composition of claim 6, wherein said non-Aciduliprofundum boonei sequence is a purification tag.

8. The pharmaceutical composition of claim 7, wherein said purification tag is a 6×-His tag.

9. The pharmaceutical composition of claim 1, further comprising a distinct anti-bacterial agent.

10. The pharmaceutical composition of claim 9, wherein said distinct anti-bacterial agent is a second anti-bacterial peptide or a chemical antibiotic.

11. A nucleic acid encoding (a) an expression cassette encoding an Aciduliprofundum boonei glycosyl hydrolase 25 muramidase (GH25) domain, operably linked to a promoter, or (b) a replicable vector encoding Aciduliprofundum boonei glycosyl hydrolase 25 muramidase (GH25) domain.

12-25. (canceled)

26. A cell comprising a nucleic acid segment encoding an Aciduliprofundum boonei glycosyl hydrolase 25 muramidase (GH25) domain, wherein said cell is not an Aciduliprofundum boonei cell.

27-30. (canceled)

31. A method of inhibiting a bacterium comprising contacting said bacterium with an Aciduliprofundum boonei glycosyl hydrolase 25 muramidase (GH25) domain.

32-35. (canceled)

36. The method of claim 31, further comprising contacting said bacterium with a distinct anti-bacterial agent.

37. The method of claim 31, wherein said distinct anti-bacterial agent is a second anti-bacterial peptide or a chemical antibiotic.

38-42. (canceled)

43. The method of claim 31, wherein said bacterium is located in a biological material ex vivo.

44. The method of claim 31, wherein said bacterium is located in a living animal subject.

45. The method of claim 44, wherein said living subject is a human.

46. The method of claim 44, wherein said living subject is a non-human animal.

47-50. (canceled)


SEQUENCE LISTING (19 sequences)

SEQ ID NO: 1 - protein, 186 aa, Aciduliprofundum boonei
KGIDVSHWQGNVNWTKVKNSGIIFAFVKATEGTSYVDPDFEENMEKAHASGLYVGAYHFAEPENYNAKEAAEHFVDTIKTYLKSGYLRPVLDLEEGSSLGKESLSSWVNEFMIEVFNLTGIKPIIYTNPNYAENYLDSSVSQWNLWIANYRVSSPSTGIWDSWAFWQYTDEGNVSGVSGNVDMDYY

SEQ ID NO: 2 - DNA, 558 bp, Aciduliprofundum boonei
aagggtatagatgtttcccactggcagggaaatgtgaactggactaaagttaaaaattccgggataatctttgcatttgtaaaggccacagaggggacatcttatgtggacccggattttgaagaaaatatggagaaggcacacgcatcaggattgtatgttggtgcctatcactttgcagaacctgaaaattataatgcaaaagaggccgcagagcattttgtagataccataaaaacatatctgaagagtggatatttgagacccgttttggatttagaagaaggatcatccttaggaaaagagagcttatcaagttgggtaaatgaatttatgattgaagtattcaatttaacgggtataaagccaataatttacacgaatccaaattatgctgaaaactaccttgattcctccgtatctcaatggaatctctggatagccaattatagagtttcgtccccttccacgggaatttgggactcctgggcattctggcagtacacagacgagggaaatgtatcgggggtgagtggcaatgtggatatggattattac

SEQ ID NO: 3 - protein, 574 aa, Aciduliprofundum boonei
MKRPIVWFVVFILILNAIFFAIGYGSNQKESLYFKTSSSIKGIDVSHWQGNVNWTKVKNSGIIFAFVKATEGTSYVDPDFEENMEKAHASGLYVGAYHFAEPENYNAKEAAEHFVDTIKTYLKSGYLRPVLDLEEGSSLGKESLSSWVNEFMIEVFNLTGIKPIIYTNPNYAENYLDSSVSQWNLWIANYRVSSPSTGIWDSWAFWQYTDEGNVSGVSGNVDMDYYNGNLKSLIDNFVIGGSCTLPYYDRAGALGYAYKWYSSDNSHYQKFSNSSEESTNFVSQVLIAGGISLWRGYDGKGDGALNYNGSMINPKYLNENLREYQNAKFSYYLASNFTVPEWIEKGDVVIFGDSNGEHYIYAGIVTYRNEDNLYIATHSPDEWNVSISSFFPSKYDLVNFYHIPNGTKKFMQVFRVTATALNIRTGPSTSYGIIGTVPENQEFVAYNYSIDSSGRKWWQFFYDDRVGWCAAWYTEMAYSDIFVVNVSSSLHVRSGAGTSNTILGSVYDGMLFAKKGQKYNSDEDITWYEIYWENKSAWIAGNYANYVPEFDIPYFLFIILLLAILAFKRKFNGD

SEQ ID NO: 4 - DNA, 1725 bp, Aciduliprofundum boonei
atgaaaaggccaatagtttggtttgtggtattcatactgattcttaatgctatattttttgccataggttatggtagcaatcaaaaagagagtttatatttcaaaacatcctccagcattaagggtatagatgtttcccactggcagggaaatgtgaactggactaaagttaaaaattccgggataatctttgcatttgtaaaggccacagaggggacatcttatgtggacccggattttgaagaaaatatggagaaggcacacgcatcaggattgtatgttggtgcctatcactttgcagaacctgaaaattataatgcaaaagaggccgcagagcattttgtagataccataaaaacatatctgaagagtggatatttgagacccgttttggatttagaagaaggatcatccttaggaaaagagagcttatcaagttgggtaaatgaatttatgattgaagtattcaatttaacgggtataaagccaataatttacacgaatccaaattatgctgaaaactaccttgattcctccgtatctcaatggaatctctggatagccaattatagagtttcgtccccttccacgggaatttgggactcctgggcattctggcagtacacagacgagggaaatgtatcgggggtgagtggcaatgtggatatggattattacaatggaaatttaaaatccctcatagataattttgttataggtggctcgtgtactctcccctattatgatagagcgggtgctttaggttatgcttataagtggtactcttccgacaattcccattaccagaaattttcaaactccagcgaggagagcacaaatttcgtttctcaggtattgatagcaggaggaatatctctctggagaggatatgatggtaaaggcgatggggctttaaattacaatggctcaatgataaatccaaagtatctgaatgagaatctcagagagtatcagaatgcaaaattttcgtactatcttgcttcaaattttacagttcctgaatggatagaaaaaggggatgtggtcatatttggagactcaaatggtgagcattacatatatgcaggtatagtaacatataggaatgaagacaatctttacatagccactcattctcccgatgaatggaatgtatccatatcctcgtttttcccatctaagtatgaccttgtaaatttttaccatataccgaatggaaccaagaaatttatgcaggtatttcgagttactgccactgctctcaatattcgaacaggtccaagcacgagttatggcataataggaactgtgccagagaatcaagagttcgttgcatacaattatagcattgattcatcagggcgcaaatggtggcagttcttctacgatgacagggtgggttggtgtgctgcatggtacactgaaatggcatattctgatatatttgtagtaaatgtctcctcctccctgcatgttcgctctggggctggaacatctaacaccatacttggctcagtttatgatggaatgctctttgccaagaagggacagaaatataacagcgatgaggacatcacctggtacgagatatactgggaaaacaaaagtgcttggattgcgggaaactatgcaaattatgtaccagaatttgacatcccatattttctatttattatacttctcttagcaatactcgcattcaagagaaaatttaatggagattaa

SEQ ID NO: 5 - protein, 113 aa, Artificial sequence
  Synthetic concensus polypeptide
KATEGXXXBXXXXXXXXXXXXXGXXXGAYHFXXXXXXXXQAXXFXXXXXXXXLXXXLDXEXXXXXXXXXXXXXXXXTXPXYXXXXXXXXXXXXPLWXAXXXXXXXXWQXXXXX

SEQ ID NO: 6 - DNA, 23 bp, Artificial sequence
  Synthetic primer
atggacgtaagtagctaccaagg

SEQ ID NO: 7 - DNA, 19 bp, Artificial sequence
  Synthetic primer
tcagcctttggcgagcttc

SEQ ID NO: 8 - DNA, 18 bp, Artificial sequence
  Synthetic primer
atgtktcccactggcagg

SEQ ID NO: 9 - DNA, 21 bp, Artificial sequence
  Synthetic primer
ccaccctgtcatcgtagaaga

SEQ ID NO: 10 - DNA, 23 bp, Artificial sequence
  Synthetic primer
ctytggggagcataycattttgg

SEQ ID NO: 11 - DNA, 25 bp, Artificial sequence
  Synthetic primer
ttttwccatckgtrtaytgccataa

SEQ ID NO: 12 - DNA, 22 bp, Artificial sequence
  Synthetic primer
gggtgcctctcctccaatcccc

SEQ ID NO: 13 - DNA, 23 bp, Artificial sequence
  Synthetic primer
ccactcacccccgatacatttcc

SEQ ID NO: 14 - DNA, 24 bp, Artificial sequence
  Synthetic primer
atggcgtttcattgcttgatcttt

SEQ ID NO: 15 - DNA, 25 bp, Artificial sequence
  Synthetic primer
gttgtaacatttttgcgctggagta

SEQ ID NO: 16 - DNA, 25 bp, Artificial sequence
  Synthetic primer
tcccactggcagggaaatgtgaact

SEQ ID NO: 17 - DNA, 24 bp, Artificial sequence
  Synthetic primer
atcctgatgcgtgtgccttctcca

SEQ ID NO: 18 - DNA, 24 bp, Artificial sequence
  Synthetic primer
tgttcatcggccatgttgaccacg

SEQ ID NO: 19 - DNA, 26 bp, Artificial sequence
  Synthetic primer
gctctttccgagtttctctgcctcct